(19) United States
(12) Patent Application Publication
(10) Pub. No.: US 2025/0090447 A1
(43) Pub. Date: Mar. 20, 2025

(54) NEW COSMETIC COMPOSITIONS CONTAINING ALOE VERA JUICE

(71) Applicant: YODI SAS, Paris (FR)

(72) Inventor: Hélène AZANCOT, Neuilly sur seine (FR)

(73) Assignee: YODI SAS, Paris (FR)

(21) Appl. No.: 18/579,981

(22) PCT Filed: Jul. 13, 2022
(86) PCT No.: PCT/EP2022/069563

§ 371 Date: Jan. 17, 2024

(30) Foreign Application Priority Data
Jul. 19, 2021 (FR) 2107769

(51) Int. Cl. A61K 8/9794; A61K 8/34; A61K 8/44; A61K 8/92; A61Q 19/00

(52) U.S. Cl. CPC A61K 8/9794; A61K 8/345; A61K 8/44; A61K 8/922; A61Q 19/00

(57) ABSTRACT

New cosmetic compositions and the use thereof. The compositions, which are based on aloe vera juice, arginine and at least one oil, have no added water but nevertheless exhibit rheological and sensory properties compatible with use in cosmetics. The cosmetic composition may be in the form of a cream, and may be used for skin care, including face, hair and or scalp care.

The present invention relates to new cosmetic compositions. The invention also relates to their use.

Aloe vera is a commercially very important crop and is used in food, pharmaceutical and cosmetic applications. Aloe vera juices and gels are particularly known to have beneficial properties for the skin and hair, for example in healing, as an aid against skin aging, or as a humectant.

However, although aloe vera juice naturally includes water, the cosmetic compositions currently on the market include more water, voluntarily added to the compositions, than that naturally present in the juice, which makes them very diluted compositions. This added water makes it possible to obtain the rheological and sensory properties desired for a cosmetic composition. Thus, the compositions currently on the market often include more than 50% water by weight.

However, the water added in the cosmetics industry is water that has undergone chemical purification stages.

In addition, the addition of water leads to “heavy” products, having a negative impact on the cost of transport from the production unit to the customer, but also on the environment, linked to the fact that water sweet is becoming more and more rare on earth.

Finally, the presence of added water leads to a dilution of the active ingredients, which leads to a cosmetic product diluted in active ingredients.

To overcome these problems, there is a need to prepare cosmetic compositions free of added water, but which nevertheless have rheological and sensory properties in line with consumer expectations, and which are concentrated in active ingredients due to the absence of dilution.

### BACKGROUND OF THE INVENTION

One of the aims of the invention is to provide a new association comprising aloe vera juice, without added water.

Another aim of the invention is a new cosmetic composition of aloe vera juice, free of added water.

Another aim of the invention is to be able to use the new association, or a composition comprising it as a cosmetic composition.

Another aim of the invention is to provide a natural cosmetic composition, free of products obtained by synthesis.

The inventors began in-depth research in order to develop cosmetic compositions, having properties, in particular sensory properties, interesting, in particular with regard to good rinsability, good detangling power of the hair, a feeling of softness on the skin and hair, rapid and melting absorption on the skin and hair and ease of application. These compositions, although concentrated in active ingredients, are free of preservatives of non-natural origin other than pentylene glycol and may be free of preservatives of natural origin.

A first object of the present invention is an association consisting of the following two components:
aloe vera juice stabilized with pentylene glycol, and
at least one oil,
   said association being free of added water.

According to the present invention, “components” means the 3 components stated above, namely: aloe vera juice stabilized with pentylene glycol, arginine and at least one oil.

“Aloe vera juice” is a juice contained in the leaves of a plant of the same name. The juice is typically isolated by cutting the tip of the leaf and then pressing the leaf.

In the context of the present invention, this concerns in particular a juice corresponding to the INCI name “aloe Barbadensis Leaf Juice”. The isolated juice is unstable, since certain components contained therein degrade upon contact with ambient oxygen. Thus, the juice used in the association and the cosmetic composition according to the present invention is stabilized by pentylene glycol, in particular at approximately 7% by weight.

The aloe vera used in the present invention is in particular the product “aloe vera pure organic juice 1:1 fair trade PentG (7%)”, marketed by the company Greentech (INCI Aloe Barbadensis Leaf Juice, Pentylene Glycol). The process for obtaining this commercial product consists of clarification of pure native aloe vera juice, isolated from the plant, followed by the addition of pentylene glycol, and a filtration.

By “added water” it is meant water, added by the producer of the association or cosmetic composition according to the present invention. Added water does not correspond to the water found in commercial products, i.e. in the components and ingredients, mixed to form the association or composition of the Invention.

By “free of added water” it must therefore be understood that the association or cosmetic composition according to the invention only includes the water which was initially present in the components and ingredients used in its preparation, said components and ingredients not also including no added water.

However, there is a possibility of the presence of water having been added independently of the formulator, for example by absorption of atmospheric water by the composition, components and/or ingredients.

The association or cosmetic composition according to the invention comprises 0% water added intentionally.

The expression “association” is synonymous with “association”. It should be noted that the components can be presented in separate form to be subsequently mixed together and form the association or cosmetic composition of the invention.

According to a particular embodiment, the present invention relates to an association consisting of the following 3 components:
aloe vera juice stabilized with pentylene glycol,
arginine, and
at least one oil,
   said association being free of added water.

The arginine used in the present invention is in particular L-arginine, in non-salified form. Arginine is, for example, “c grade” arginine, which comes in the form of a white powder.

According to another particular embodiment, the present invention relates to an association consisting of the following 4 components:
aloe vera juice stabilized with pentylene glycol,
arginine,
at least one oil, and
a flower acid,
   said association being free of added water.

The flower acids used in the present invention contain a-hydroxy acids, or AHAs. Among the flower acids these include red flower acids (INCI Hibiscus sabdariffa flower extract, maltodextrin).

According to another particular embodiment, the present invention relates to an association consisting of the following 4 components:
aloe vera juice stabilized with pentylene glycol,
arginine,
at least one oil, and
a prebiotic,
   said association being free of added water.

Among the prebiotics, these include inulin and the α glucan oligosaccharide.

Particularly advantageously, the prebiotic is the glucan oligosaccharide.

Among the a-glucan oligosaccharide that can be used and commercially available, these include Bioecolia® (INCI: a-glucan oligosaccharide) or Ecoskin® (INCI: Alpha-glucan oligosaccharide, Polymnia sonchifolia root juice, Maltodextrin, Lactobacillus) from the company Solabia, or Biolin/P (INCI: Inulin (and) Alpha-Glucan Oligosaccharide) from the company Gobiotics.

According to another particular embodiment, the present invention relates to an association consisting of the following 4 components:
aloe vera juice stabilized with pentylene glycol,
arginine,
at least one oil, and
glyceryl stearate,
   said association being free of added water.

According to another particular embodiment, the present invention relates to an association as defined above, further comprising calcium gluconate.

The synergy from the different components gives rise to an association whose use in a cosmetic composition confers interesting properties, particularly in relation to sensory properties.

The association, and the cosmetic composition according to the present invention are made up of natural components and ingredients, or of natural origin, and of non-animal origin, in particular vegan and of plant origin.

According to another particular embodiment, the present invention relates to an association as defined above, in which the at least one oil is chosen from:
jojoba oil,
sweet almond oil,
oil of bitter almond,
argan oil,
sesame oil,
camellia oil,
safflower oil,
moringa oil,
castor oil,
coconut oil,
apricot oil,
flaxseed oil,
pomegranate oil,
grapeseed oil,
prickly pear oils,
marula oil,
evening primrose oil,
avocado oil,
raspberry oil,
borage oil,
olive oil,
neem oil,
sunflower oil,
walnut or hazelnut oil,
amla oil,
plum oil,
coffee oil,
hemp oil,
rice bran oil,
vanilla macerate,
oil powder, and
fermented oil,
or a mixture thereof.

By “at least one oil” it is meant in particular one oil, or two different oils. According to a preferred embodiment, the oil is argan oil, or a mixture of argan oil and sunflower oil.

By “oil powder” it is meant oils that are initially liquid at room temperature, but have been formulated to be solid at room temperature. Oils can, for example, be encapsulated to produce microcapsules in powder form. Encapsulating agents include alginate, maltodextrin, arrowroot powder, or corn starch.

Among the oils that can be formulated in powder form, these include vegetable oils, in particular oils as defined above.

These include argan oil powder (argan oil coated with maltodextrin), jojoba powder, castor oil powder, pineapple oil powder, or sweet almond oil powder.

By “fermented oil” it is meant an oil that has undergone a fermentation process. Among the oils capable of being fermented, these include vegetable oils, in particular oils as defined above. These include in particular fermented jojoba oil, fermented argan oil, fermented moringa oil, fermented sesame oil, fermented sweet almond oil, fermented hemp oil, fermented safflower oil, fermented camellia oil, or fermented olive oil.

According to another particular embodiment, the present invention relates to an association as defined above, in which the oil is an oil powder as defined above.

According to another particular embodiment, the present invention relates to an association as defined above, in which the oil is a fermented oil as defined above.

According to another particular embodiment, the present invention relates to an association as defined above, in which the quantity of aloe vera juice stabilized by pentylene glycol is from 60 to 95%, as a percentage by weight relative to the total weight of the association.

In relation to the quantity of aloe vera juice stabilized with pentylene glycol, “from 60 to 95%” also means the following ranges: from 60 to 90%, from 60 to 80%, from 60 to 70%, from 70 to 95%, from 80 to 95%, from 70 to 90%, from 75 to 85%.

In another particular embodiment, the present invention relates to an association as defined above, in which the quantity of aloe vera juice is from 56 and 88%, as a percentage by weight relative to the total weight of the ‘association.

In relation to the quantity of aloe vera juice, “from 56 to 88%” also means the following ranges: from 56 to 85%, from 56 to 80%, from 56 to 70%, from 60 to 88%, from 70 to 88%, from 80 to 88%, from 70 to 80%, from 75 to 85%.

In another particular embodiment, the present invention relates to an association as defined above, in which the quantity of pentylene glycol is from 4 and 7%, as a percentage by weight relative to the total weight of the association.

In relation to the quantity of pentylene glycol, “from 4 to 7%” also means the following ranges: from 4 to 5%, from 4 to 6%, from 5 to 6%, from 6 to 7%, from 5 at 7%.

According to another particular embodiment, the present invention relates to an association as defined above, in which the quantity of arginine is comprised from 0.00015 to 5%, as a percentage by weight relative to the total weight of the association.

In relation to the quantity of arginine, “from 0.00015 to 5%” also means the following ranges: from 0.00015 to 4%, from 0.00015 to 2%, from 0.00015 to 1%, from 0.00015 to 0.1%, from 0.00015 to 0.01%, from 0.001 to 5%, from 0.01 to 5%, from 0.1 to 5%, from 1 to 5%, from 2 at 5%, from 4 to 5%, from 0.01 to 4%, from 0.1 to 3%, from 1 to 2%.

According to another particular embodiment, the present invention relates to an association as defined above, in which the total quantity of oil is from 3 and 40%, as a percentage by weight relative to the total weight of the association.

In relation to the total quantity of oil, “from 3 to 40%” also means the following ranges: from 3 to 30%, from 3 to 20%, from 3 to 10%, from 10 to 40%, from 20 to 40%, 10 to 40%, 20 to 30%.

In another particular embodiment, the present invention relates to an association as defined above, said association consisting of:
aloe vera juice stabilized by pentylene glycol, the quantity of aloe vera juice stabilized by pentylene glycol is from 60 to 95%, as a percentage by weight relative to the total weight of the association,
at least one oil,
   said association being free of added water.

In another particular embodiment, the present invention relates to an association as defined above, said association consisting of:
aloe vera juice stabilized by pentylene glycol,
at least one oil, the quantity of oil being included from 3 to 40%, as a percentage by weight relative to the total weight of the association,
   said association being free of added water.

In another particular embodiment, the present invention relates to an association as defined above, said association consisting of: aloe vera juice stabilized by pentylene glycol, the quantity of aloe vera juice stabilized by pentylene glycol being from 60 to 95%, as a percentage by weight relative to the total weight of the association, at least one oil, the quantity of oil being from 3 to 40%, as a percentage by weight relative to the total weight of the association, said association being free of added water.

In another embodiment, the present invention relates to an association as defined above, said association consisting of:
aloe vera juice, the quantity of aloe vera juice being from 56 to 88%, as a percentage in weight relative to the total weight of the association,
pentylene glycol,
at least one oil,
   said association being free of added water.

In another embodiment, the present invention relates to an association as defined above, said association consisting of:
aloe vera juice,
pentylene glycol, the quantity of pentylene being from 4 to 7%, as a percentage by weight relative to the total weight of the association,
at least one oil,
   said association being free of added water.

In another embodiment, the present invention relates to an association as defined above, said association consisting of:
aloe vera juice,
pentylene glycol,
at least one oil, the quantity of oil being comprised from 3 to 40%, as a percentage by weight relative to the total weight of the association,
   said association being free of added water.

In another embodiment, the present invention relates to an association as defined above, said association consisting of:
aloe vera juice, the quantity of aloe vera juice being from 56 to 88%, as a percentage by weight relative to the total weight of the association,
pentylene glycol, the quantity of pentylene being from 4 to 7%, as a percentage by weight relative to the total weight of the association,
at least one oil,
   said association being free of added water.

In another embodiment, the present invention relates to an association as defined above, said association consisting of:
aloe vera juice, the quantity of aloe vera juice being from 56 to 88%, as a percentage by weight relative to the total weight of the association,
pentylene glycol,
at least one oil, the quantity of oil being from 3 to 40%, as a percentage by weight relative to the total weight of the association,
   said association being free of added water.

In another embodiment, the present invention relates to an association as defined above, said association consisting of:
aloe vera juice,
pentylene glycol, the quantity of pentylene being from 4 to 7%, as a percentage by weight relative to the total weight of the association,
at least one oil, the quantity of oil being from 3 to 40%, as a percentage by weight relative to the total weight of the association,
   said association being free of water added.

In another embodiment, the present invention relates to an association as defined above, said association consisting of:
aloe vera juice, the quantity of aloe vera juice being from 56 to 88%, as a percentage by weight relative to the total weight of the association,
pentylene glycol, the quantity of pentylene being from 4 to 7%, as a percentage by weight relative to the total weight of the association,
at least one oil, the quantity of the oil being comprised from 3 to 40%, as a percentage by weight relative to the total weight of the association,
   said association being free of added water.

A second object of the present invention concerns the use of an association as defined above, in a cosmetic composition.

A third object of the present invention relates to a cosmetic composition, comprising an association as defined above, in a cosmetically acceptable medium, said cosmetic composition being free of added water.

By “a cosmetically acceptable medium”, it is meant within the meaning of the invention a medium compatible with use in cosmetics.

According to a particular embodiment, the present invention relates to a cosmetic composition as defined above, comprising, in a cosmetically acceptable medium, the following 2 components:
aloe vera juice stabilized with pentylene glycol, and
at least one oil,
   said cosmetic composition being free of added water.

According to another particular embodiment, the present invention relates to a cosmetic composition as defined above, comprising, in a cosmetically acceptable medium, the following 3 components:
aloe vera juice stabilized with pentylene glycol,
arginine, and
at least one oil,
   said cosmetic composition being free of added water.

According to another particular embodiment, the present invention relates to a cosmetic composition as defined above, comprising, in a cosmetically acceptable medium, the following 4 components:
aloe vera juice stabilized with pentylene glycol,
arginine,
at least one oil, and
vitamin E,
   said cosmetic composition being free of added water.

According to another particular embodiment, the present invention relates to a cosmetic composition as defined above, comprising, in a cosmetically acceptable medium, the following 4 components:
aloe vera juice stabilized with pentylene glycol,
arginine,
at least one oil, and
a prebiotic, in particular glucan oligosaccharide,
   said cosmetic composition being free of added water,
   said cosmetic composition being a composition for the care of the skin, in particular the face.

Among the skin care products these include a nourishing treatment, a moisturizing treatment, an anti-wrinkle treatment and a tightening treatment.

According to another particular embodiment, the present invention relates to a cosmetic composition as defined above, comprising, in a cosmetically acceptable medium, the following 4 components:
aloe vera juice stabilized with pentylene glycol,
arginine,
at least one oil, and
a flower acid,
   said cosmetic composition being free of added water,
   said cosmetic composition being a composition for hair care.

Among the hair care products these include: rinse-out hair care, a hair mask, in particular to rinse out, a rinse-out detangling treatment, a nourishing and moisturizing rinse-out treatment, a leave-in treatment.

According to another particular embodiment, the present invention relates to a cosmetic composition as defined above, further comprising calcium gluconate.

The calcium gluconate, present in the cosmetic composition according to the invention, is in particular derived from a commercial form of the surfactant “brassicyl valinate esylate” which is marketed under the name AminoSensyl™ HC. This commercial product indeed includes: brassicyl valinate esylate, calcium gluconate and Brassica alcohol.

According to another particular embodiment, the present invention relates to a composition as defined above, in which the quantity of aloe vera juice stabilized by pentylene glycol is from 45 and 80%, as a percentage by weight relative to the total weight of the composition.

In relation to the quantity of aloe vera juice stabilized with pentylene glycol, “from 45 to 80%” also means the following ranges: from 50 to 80%, from 60 to 80%, from 70 to 80%, from 45 to 70%, from 45 to 60%, from 45 to 50%, from 50 to 75%, from 60 to 75%.

The quantity of aloe vera stabilized by pentylene glycol is notably from 70 and 75%.

In another particular embodiment, the present invention relates to a composition as defined above, in which the quantity of aloe vera juice is from 42 and 74.2%, as a percentage by weight relative to the total weight. of the composition.

In relation to the quantity of aloe vera juice, “from 42 to 74%” also means the following ranges: from 42 to 70%, from 42 to 60%, from 42 to 50%, from 50 to 74, 2%; from 60 to 74.2%, from 70 to 74.2%.

In another particular embodiment, the present invention relates to a composition as defined above, in which the quantity of pentylene glycol is from 3 and 5.8%, as a percentage by weight relative to the total weight of the composition.

In relation to the quantity of pentylene glycol, “from 3 to 5.8%” also means the following ranges: from 3 to 5%, from 3 to 4%, from 4 to 5.8%, from 4 to 5%. According to another particular embodiment, the present invention relates to a composition as defined above, in which the quantity of arginine is from 0.0001 to 2%, as a percentage by weight relative to the total weight of the composition.

“From 0.0001 to 2%” also means the following ranges: from 0.001 to 2%, from 0.01 to 2%, from 0.1 to 2%, from 0.5 to 2%, from 1 to 2%, from 0.0001 to 1.5%, from 0.0001 to 1%, from 0.0001 to 0.5%, from 0.0001 to 0.1%, from 0.0001 to 0.01%, from 0.01 to 1%, from 0.1 to 0.5%.

The quantity of arginine is notably around 0.0001% or around 1.5%.

According to another particular embodiment, the present invention relates to a composition as defined above, in which the total quantity of oil is from 2 and 25%, as a percentage by weight relative to the total weight of the composition.

“From 2 to 25%” also means the following ranges: from 5 to 25%, from 10 to 25%, from 15 to 25%, from 2 to 20%, from 2 to 15%, from 2 to 10%, from 2 to 5%, from 5 to 15%.

According to another particular embodiment, the present invention relates to a composition as defined above, in which the total quantity of oil is from 1 and 25%, as a percentage by weight relative to the total weight of the composition.

The total quantity of oil is in particular from 1 to 10%, 2 to 10% or 15 to 25%.

In particular, for skin care, the range of 15 to 25% is preferred, while in the context of hair care, the range of 1 to 10% or 2 to 10% is preferred.

In another particular embodiment, the present invention relates to a cosmetic composition as defined above, said composition comprising, in a cosmetically acceptable medium:
aloe vera juice stabilized with pentylene glycol, the quantity of juice aloe vera stabilized with pentylene glycol is from 45 to 80%, as a percentage by weight relative to the total weight of the composition,
at least one oil,
   said cosmetic composition being free of added water.

In another particular embodiment, the present invention relates to a cosmetic composition as defined above, said composition comprising, in a cosmetically acceptable medium:
aloe vera juice stabilized with pentylene glycol,
at least one oil, quantity of oil being comprised from 1 to 25% or from 2 to 25%, as a percentage by weight relative to the total weight of the composition,
   said cosmetic composition being free of added water.

In another particular embodiment, the present invention relates to a cosmetic composition as defined above, said composition comprising, in a cosmetically acceptable medium:
aloe vera juice stabilized with pentylene glycol, the quantity of juice of aloe vera stabilized with pentylene glycol being from 45 to 80%, as a percentage by weight relative to the total weight of the composition,
at least one oil, the quantity of oil being from 1 to 25% or from 2 to 25%, as a percentage by weight relative to the total weight of the composition,
   said cosmetic composition being free of added water.

In another embodiment, the present invention relates to a cosmetic composition as defined above, said composition comprising, in a cosmetically acceptable medium:
aloe vera juice, the quantity of aloe vera juice being comprised from 42 to 74.2%, as a percentage by weight relative to the total weight of the composition, of pentylene glycol,
at least one oil,
   said cosmetic composition being free of added water.

In another embodiment, the present invention relates to a cosmetic composition as defined above, said composition comprising, in a cosmetically acceptable medium:
aloe vera juice, pentylene glycol, the quantity of pentylene being comprised from 3 to 5.8%, as a percentage by weight relative to the total weight of the composition,
at least one oil,
   said cosmetic composition being free of added water.

In another embodiment, the present invention relates to a cosmetic composition as defined above, said composition comprising, in a cosmetically acceptable medium:
aloe vera juice, pentylene glycol,
at least one oil, the quantity of the oil being comprised from 1 to 25% or from 2 to 25%, as a percentage by weight relative to the total weight of the composition,
   said cosmetic composition being free of added water.

In another embodiment, the present invention relates to a cosmetic composition as defined above, said composition comprising, in a cosmetically acceptable medium:
aloe vera juice, the quantity of aloe vera juice being comprised from 42 to 74.2%, as a percentage by weight relative to the total weight of the composition, of pentylene glycol, the quantity of pentylene being from 3 to 5.8%, as a percentage by weight relative to the total weight of the composition,
at least one oil,
   said cosmetic composition being free of added water.

In another embodiment, the present invention relates to a cosmetic composition as defined above, said composition comprising, in a cosmetically acceptable medium:
aloe vera juice, the quantity of aloe vera juice being comprised from 42 to 74.2%, as a percentage by weight relative to the total weight of the composition, of pentylene glycol,
at least one oil, the quantity of oil being comprised from 1 to 25% or from 2 to 25%, as a percentage in weight relative to the total weight of the composition,
   said cosmetic composition being free of added water.

In another embodiment, the present invention relates to a cosmetic composition as defined above, said composition comprising, in a cosmetically acceptable medium:
aloe vera juice,
pentylene glycol, the quantity of pentylene being from 3 to 5.8%, as a percentage by weight relative to the total weight of the composition,
at least one oil, the quantity of oil being from 1 to 25% or 2 to 25%, as a percentage by weight relative to the total weight of the composition,
   said cosmetic composition being free of added water.

In another embodiment, the present invention relates to a cosmetic composition as defined above, said composition comprising, in a cosmetically acceptable medium:
aloe vera juice, the quantity of aloe vera juice being comprised of 42 at 74.2%, as a percentage by weight relative to the total weight of the composition,
pentylene glycol, the quantity of pentylene being from 3 to 5.8%, as a percentage by weight relative to the total weight of the composition,
at least one oil, the quantity of oil being comprised from 1 to 25% or from 2 to 25%, as a percentage by weight relative to the total weight of the composition,
   said cosmetic composition being free of added water.

According to another particular embodiment, the present invention relates to a composition as defined above, said composition also comprising arginine.

According to another particular embodiment, the present invention relates to a composition as defined above, in which the quantity of arginine is from 0.00015 to 5%, as a percentage by weight relative to the total weight of the composition.

According to another particular embodiment, the present invention relates to a composition as defined above, in which the composition further comprises at least one ingredient, in particular an active compound, in particular chosen from:
a surfactant,
glycerin,
cannabidiol,
flower acid,
Zinc PCA,
a pigment,
vegetable charcoal,
an antioxidant, in particular vitamin C, or one of its derivatives,
niacinamide,
powdered tea, in particular matcha green tea, fermented tea, powdered kombucha tea, centella asiatica, or madecassoside,
sugars, in particular xylitol, rhamnose, or fructooligosaccharides, apple extract powder a protein, in particular pea protein, soy protein, wheat protein, or rice protein, sweet almond milk, or one or more of its components, in particular prunus dulcis, rice syrup or acacia fiber,
a fragrance, in particular an apricot almond fragrance, a coconut milk fragrance, ghassoul, white clay, in particular kaolin,
hyaluronic acid or one of these derivatives, in particular sodium hyaluronate, lactic acid, vanilla macerate,
maltodextrin,
bamboo powder,
spirulina,
phycocianin,
fermented mushrooms,
guarana powder,
caffeine,
acerola,
chamomile,
ayurvedic powders,
vitamins, in particular vitamin B3, or vitamin B5,
glycosphingolipids and glycolipids,
a prebiotic, exopolysaccharides, postbiotics, in particular lactobacilus ferment, pyroctolamine,
piroctone olamine,
an extract of camellia seeds,
powder of cornflower,
chamomile flower extract,
calcium gluconate,
collagen,
elastin,
Manuka honey,
vinegar, especially rice vinegar,
sodium phytate,
ceramide,
probiotic,
marshmallow powder, and
peptides, especially quinoa peptides, or
a mixture of these ingredients.

The total weight of said at least one ingredient is in particular comprised from 0 to 30% by mass relative to the total weight of the composition, in particular from 20 to 25% or from 5 to 10%.

According to the present invention, “ingredient” means the compounds, which may optionally be contained, other than the 3 or 4 components.

The ingredients, but also possibly the components, have either an activity on the skin, or on the hair, or properties at the sensorial level.

Thus, the cosmetic compositions of the present invention comprise substances having various properties, such as moisturizing, lightening, purifying, anti-aging, anti-wrinkle, energizing, anti-dark circles and/or anti-sebum properties.

The surfactants are in particular non-sulphated surfactants. Among the surfactants these include, by way of example, lauryl glucoside, decyl glucoside, caprylyl/capryl glucoside, coco-glucoside, hexyl glucoside, sodium cocoyl glutamate, disodium cocoyl glutamate, sodium stearoyl glutamate, sodium lauroyl glutamate, sodium myristoyl glutamate, coco-betaine, cocamidopropyl betaine, disodium cocoamphodiacetate, sodium amphoacetate, sodium babassuamphoacetate, sodium cocoabutteramphoacetate, sodium cottonseedamphoacetate, sodium sheabutteramphoacetate, sodium mangoamphoacetate, sodium olivamphoacetate, sodium ricebranamphoacetate, sodium sweet almondamphoacetate, sodium sunflowerseedamphoacetate, potassium cocoate, potassium palmate, potassium laurate, potassium olivate, sodium coco-sulfate, sodium cocoamphoacetate, disodium coco-glucoside citrate, sodium coco-glucoside tartrate, sodium lauroamphoacetate, cocoyl methyl glucamide, capryloyl/caproyl methyl glucamide, lauroyl/myristoyl methyl glucamide, ammonium lauryl sulfate, ammonium coco-sulfate, magnesium lauryl sulfate, sodium C12-18 alkyl sulfate, zinc coco-sulfate, sodium cocoyl alaninate, sodium lauryl sulfate, sodium cetearyl sulfate, sodium cocoyl isethionate, sodium methyl oleoyl taurate, sodium methyl cocoyl taurate, cocamide MEA, cocamide MIPA, sodium C14-16 olefin sulfonate, sodium lauryl sulfoacetate, sodium lauroyl sarcosinate, sodium laureth sulfate, ammonium laureth sulfate, cocamidopropyl hydroxysultaine, disodium laureth sulfosuccinate, disodium lauryl sulfosuccinate, sodium myreth sulfate, laureth-5 carboxylic acid, glyceryl stearate, brassicyl valinate esylate, and saponin.

“Zinc PCA” is a zinc salt of pyrrolidone carboxylic acid, which is marketed for example under the name Zincidone®.

Among the pigments, these include Unicert violet K7025-J (INCI: Cl 60730), comprising the benzene sulfonic acid dye 2-[(9,10-dihydro-4-hydroxy-9,10-dioxo-1-anthracenyl)amino]-5-methyl monosalt sodium (CAS 4430-18-6).

Among the “exopolysaccharides” these include:

the isomerate saccharide, for example:
from the Epidermist™ 4.0 brand (INCI: Aqua, Saccharide Isomerate),
from the EPS Seamat™ brand (INCI: Aqua, Saccharide Isomerate),
from the EPS seapur® brand (INCI: Water, Phenethyl alcohol, Saccharide isomerate),
from the EPS seaglow® brand (INCI: Water, Saccharide isomerate, Sodium benzoate, Citric acid),
from the Actibiome brand from the company Codif (INCI: Glycerin, Water, Seawater, Laminaria digitata extract, Chlorella vulgaris extract, saccharide isomerate, phenethylalcohol),
   Alteromonas ferment extract, for example from the EPS Seafill™ brand (INCI: Aqua, Alteromonas Ferment Extract).
   or the exopolysaccharides from EPS seawhite® brand.

Among the “Ayurvedic powders” these include:
Shikakaï powder (INCI: Acacia concinna fruit powder),
Reetha powder (INCI: Sapindus mukorossi powder),
sidr (INCI: Zizyphus jujuba leaf powder),
brahmi powder (INCI: Bacopa Monnieri),
Bhringaraj Powder (INCI: Eclipta alba powder),
amla (INCI: Emblica officinalis fruit powder),
Fenugreek (methi) powder (INCI: Trigonella foenum-graecum seed powder),
Hibiscus powder (INCI: Hibiscus sabdariffa flower powder),
Kapoor Kachli (INCI: Hedychium spicatum),
Tulsi powder (INCI: Ocimum sanctum leaf powder),
Sugandhi kachur powder (INCI: Kaempferia galanga root powder),
Neem powder (INCI: Azadirachta indica leaves powder),
Nagarmotha powder (INCI: Cyperus rotundus root powder), and
Orange powder is notably a product corresponding to: INCI: Citrus aurantium peel powder.

Vanilla macerate is in particular a product corresponding to: INCI: Corylus avellana Seed Oil/Vanilla planifolia fruit.

Glycosphingolipids and glycolipids are notably of the Ceramosides™ HP brand (INCI: Glycosphingolipids, glycolipids).

Postbiotics include lactobacillus ferment, for example from the lactobiotyl® brand (INCI: Maltodextrin and Lactobacillus Ferment).

Camellia seed extract is notably a product corresponding to: INCI: Camellia oleifera seed extract.

Cornflower extract is notably a product corresponding to: INCI: Centaurea cyanus flower extract.

Chamomile flower extract is notably a product corresponding to: INCI Maltodextrin, Chamomilla Recutita Flower Extract.

Antioxidants are used to prevent the oil from going rancid. They do not affect the microbial stability of the cosmetic composition. As “antioxidant”, these include, for example, tocopherol (vitamin E) or ascorbic acid (vitamin C), or a derivative of vitamin C, in particular ascorbyl glucoside.

Rancidity is caused by the oxidation of vegetable oils and butters in contact with air. This phenomenon is accelerated by light (UV), temperature, contact with pro-oxidant metals (Iron or Copper), the presence of pigments such as chlorophyll or the presence of enzymes (lipases, etc.). Oxidation is easily identifiable because it is responsible for a change in the appearance of the vegetable oil (rancid smell, change in color).

There are several methods to determine the oxidative stability of lipids:
Measurement of absorbed oxygen: this method makes it possible to simultaneously monitor the course of oxidation and measure the oxidation index of the treated oil. Absorbed oxygen is measured by volumetric or electrochemical methods.
Oven method or Shaal test: The test consists of oxidizing the fat in a lit oven at 60° C. Evidence of oxidation is shown by measuring the peroxide index on samples taken every 4, 8, or 24 hours. This method has the advantage of being close to actual storage conditions (case of transparent bottles of oils stored in daylight and at room temperature).

Rancimat test: This test is widely used to evaluate the oxidative stability of fats. The TIR specification (Rancimat test induction time, expressed in hours) corresponds to the time during which the fat resisted oxidative stress. The principle of the test consists of prematurely aging the fats by thermal decomposition at 98° C., under intensive air bubbling. The organic acids, degradation products of this extensive oxidation, are carried away by a current of air and collected in a measuring cell filled with distilled water. The induction time is determined by conductimetry and corresponds to the TIR. The Rancimat test offers the advantage of monitoring several samples in parallel, with reduced analysis times.

In one embodiment, the invention relates to a composition as described above, said composition exhibiting an absence of rancidity of the oil.

The fragrances used in the present invention are natural fragrances or of natural origin.

These include an herbal and tea fragrance, an apricot almond fragrance, or a coconut milk fragrance, which are for example marketed by the company Technicoflor.

According to another particular embodiment, the present invention relates to a composition as defined above, said composition being free of:
nanoparticles,
synthetic preservatives,
ingredients of animal origin,
parabens,
polyethylene glycol (PEG),
sulfates,
talc,
silicones,
allergens,
synthetic fragrances,
irritating active ingredients, and
mineral salts of heavy metals.

The cosmetic composition of the present invention is in particular free of mineral salts of heavy metals.

The cosmetic compositions according to the present invention are in particular free of non-natural materials or of non-natural origin, and of animal materials.

In this context, the expression “free” means mass quantities less than 100 ppm, in particular less than 50 ppm, in particular less than 10 ppm.

Preservatives are conventionally used to prevent spoilage caused by microorganisms and therefore make it possible to guarantee the microbial stability of the cosmetic composition in which they are added, during normal use by the consumer and in order to maintain the quality and safety of the product.

Challenge Tests are necessary to ensure that microbial growth is minimized under normal conditions of storage and/or use over the expected life of the product.

The objective of the Challenge Test is to demonstrate the effectiveness of the conservation system during the shelf life and use of the cosmetic product. This is a mandatory regulatory requirement aimed at reducing risks to consumer safety.

When carrying out the Challenge Test, the formulation of the cosmetic product must resist microbial development for 28 days at 32.5° C., which makes it possible to best represent its shelf life and use in the minimum possible time. The test is carried out by the artificial introduction of known and calibrated microorganisms into a measured aliquot of the cosmetic. Then, an analysis of these is carried out on days 0, 7, 14 and 28 according to a protocol validated upstream.

The mandatory microorganisms used for the Challenge Test are: Escherichia coli, Staphylococcus aureus, Pseudomonas aeruginosa, Candida albicans and Aspergillus brasiliensis.

For the product to pass the tests, its preservation system must be effective enough to reduce the level of microorganisms to the authorized microbial limits predefined in the IS011 930 standard concerning the evaluation of the antimicrobial protection of a cosmetic product. In particular, the preservation system is considered effective when the density of germs in the product is less than 500 CFU/gram of product, preferably less than 300 CFU/gram of product. Ideally, the germ density in the product is less than 10 CFU/gram of product. The composition is then stable from a microbiological point of view. There are synthetic, non-natural preservatives and natural preservatives. Examples of synthetic preservatives include triclosan, triclocarban, phenylethanol, benzyl benzoate, methylisothiazolinone, zinc pyrithione, benzalkonium chloride, and benzyl alcohol.

Furthermore, the expression synthetic refers to non-natural products, obtained by chemical synthesis.

The association or cosmetic composition according to the present invention is free of any non-natural chemical preservative.

Examples of natural preservatives include benzoic acid, sorbic acid and salicylic acid.

The association or cosmetic composition according to the present invention is free of any natural preservative.

In one embodiment, the composition as described above is free of any synthetic preservative.

In another embodiment, the composition as described above is free of any preservative, synthetic or natural, other than pentylene glycol.

In another embodiment, the composition as described above has microbial stability.

Among the parabens these include, for example, methyl paraben, propyl paraben, butyl paraben, and ethyl paraben.

By “polyethylene glycol” it is meant a polymer consisting of ethylene glycol monomers which can have various molecular weights, for example PEG-1000 and PEG-2000.

Among the silicones these include, by way of example, dimethicone, cyclomethicone, amodimethicone, ceteraryl methicone, dimethiconol, and stearyl dimethicone.

By “allergens” it is meant substances known to be irritating or allergenic. As an example these include: Ga-Isomethyl ionone, amyl cinnamal, amylcinnamyl alcohol, anisic alcohol, benzyl alcohol, benzyl benzoate, benzyl cinnamate, salicylate of benzyl, butylphenyl methylpropional, cinnamal, cinnamyl alcohol, citral, citronellol, coumarin, eugenol, farnesol, geraniol, hexyl cinnamal, ydroxycitronnellal, isoeugenol, limonene, linalool, and 2-methyl octynoate.

By “irritant active ingredients” it is meant products presenting a risk of skin irritation.

This is any active ingredient known to have an irritant property in the field of cosmetics.

These include by way of example: sulfated surfactants, such as sodium lauryl sulfate or sodium coco sulfate, and guar hydroxypropyltrimoniumchloride.

According to another particular embodiment, the present invention relates to a composition as defined above, in which said composition comprises:
aloe vera juice,
pentylene glycol,
tara gum,
glycerin,
brassicyl valinate esylate,
Brassica alcohol,
calcium gluconate,
argan oil,
red flower acids,
fragrance of coconut milk, and
arginine.

According to another particular embodiment, the present invention relates to a composition as defined above, in which said composition comprises:
aloe vera juice
pentylene glycol
Tara gum
glycerin,
brassicyl valinate esylate,
Brassica alcohol,
argan oil
vegetable charcoal
alpha-glucan oligosaccharide
an anti-dandruff agent
herbal and tea scent

According to this particular embodiment, the anti-dandruff agent can be chosen propanediol caprylate.

The cosmetic composition according to this particular embodiment is in particular a composition for hair care, and can optionally comprise calcium gluconate.

According to another particular embodiment, the present invention relates to a composition as defined above, in which said composition comprises:
aloe vera juice,
pentylene glycol,
xanthan gum,
glyceryl stearate,
hydrogenated lecithin,
argan oil,
sunflower oil,
vitamin E,
citric acid,
a-glucan oligosaccharide, and
arginine.

According to another particular embodiment, the present invention relates to a composition as defined above, in which said composition comprises:
aloe vera juice,
pentylene glycol,
sodium phytate
brassicyl valinate esylate,
Brassica alcohol,
calcium gluconate,
argan oil,
tara gum,
glycerin,
arginine.

According to another particular embodiment, the present invention relates to a composition as defined above, in which said composition comprises:
aloe vera juice,
pentylene glycol,
glyceryl stearate,
brassicyl valinate esylate,
Brassica alcohol,
calcium gluconate,
argan oil,
tara gum,
glycerin,
arginine.

The cosmetic composition according to this particular embodiment is in particular a composition for skin care, and can optionally comprise calcium gluconate.

According to another particular embodiment, the present invention relates to a composition as defined above, said composition being in the form of a cream, in particular a melting cream.

The cosmetic compositions, as well as the association, according to the present invention are in particular “sterile”, in other words, they are free of microorganisms, in particular chosen from bacteria and fungi, such as Candida albicans, Escherichia coli, Staphylococcus aureus, Pseudomonas aeruginosa and Aspergillus brasiliensis, the concentration of microorganisms 24 hours after the preparation of the cosmetic composition, or of the association, being less than 1E+04 in number of colony-forming units.

The cosmetic compositions, as well as the association according to the present invention, are in particular stable over time, in particular at temperatures of 4, 20, 40 or 50° C., for at least one month.

By “stable over time”, it is meant an absence of coloring and/or phase separation.

In a particular embodiment, the viscosity of the cosmetic composition according to any one of the embodiments defined above is from 45,000 and 80,000 cps.

In relation to viscosity, by “from 45,000 to 80,000 cps” it is also meant the following ranges: from 50,000 to 80,000 cps, from 55,000 to 80,000 cps, from 60,000 to 80,000 cps, from 65,000 to 80,000 cps, from 70,000 to 80,000 cps, from 75,000 to 80,000 cps, from 45,000 to 75,000 cps, from 45,000 to 70,000 cps, from 45,000 to 65,000 cps, from 45,000 to 60,000 cps, from 45,000 to 55,000 cps, from 45,000 to 50,000 cps, from 60,000 to 70,000 cps, from 55,000 to 75,000 cps.

According to the present invention, the viscosity measurement is carried out using the RVDVI, mobile S06 equipment at a speed of 6 RPM for 1 minute at a temperature of 20° C. and at atmospheric pressure.

In another particular embodiment, the present invention relates to a cosmetic composition as defined above, said composition comprising, in a cosmetically acceptable medium:
aloe vera juice stabilized with pentylene glycol, the quantity of juice aloe vera stabilized with pentylene glycol is from 45 to 80%, as a percentage by weight relative to the total weight of the composition,
at least one oil,
   said cosmetic composition being free of added water,
   the viscosity of said composition being from 45,000 and 80,000 cps.

In another particular embodiment, the present invention relates to a cosmetic composition as defined above, said composition comprising, in a cosmetically acceptable medium:
aloe vera juice stabilized with pentylene glycol,
at least one oil, quantity of oil being comprised from 1 to 25% or from 2 to 25%, as a percentage by weight relative to the total weight of the composition,
   said cosmetic composition being free of added water,
   the viscosity of said composition being comprised of 45000 at 80000 cps.

In another particular embodiment, the present invention relates to a cosmetic composition as defined above, said composition comprising, in a cosmetically acceptable medium:
aloe vera juice stabilized with pentylene glycol, the quantity of juice of aloe vera stabilized with pentylene glycol being from 45 to 80%, as a percentage by weight relative to the total weight of the composition,
at least one oil, the quantity of oil being from 1 and 25% or from 2 to 25%, as a percentage by weight relative to the total weight of the composition,
   said cosmetic composition being free of added water,
   the viscosity of said composition being from 45,000 to 80,000 cps.

In another embodiment, the present invention relates to a cosmetic composition as defined above, said composition comprising, in a cosmetically acceptable medium:
aloe vera juice, the quantity of aloe vera juice being comprised of 42 at 74.2%, as a percentage by weight relative to the total weight of the composition,
pentylene glycol,
at least one oil,
   said cosmetic composition being free of added water,
   the viscosity of said composition being from 45,000 to 80,000 cps.

In another embodiment, the present invention relates to a cosmetic composition as defined above, said composition comprising, in a cosmetically acceptable medium:
aloe vera juice,
pentylene glycol, the quantity of pentylene being from 3 and 5.8%, as a percentage by weight relative to the total weight of the composition,
at least one oil,
   said cosmetic composition being free of added water,
   the viscosity of said composition being from 45,000 to 80,000 cps.

In another embodiment, the present invention relates to a cosmetic composition as defined above, said composition comprising, in a cosmetically acceptable medium:
aloe vera juice,
pentylene glycol,
at least one oil, the quantity of oil being comprised from 1 to 25% or from 2 to 25%, as a percentage by weight relative to the total weight of the composition,
   said cosmetic composition being free of added water,
   the viscosity of said composition being comprised from 45,000 to 80,000 cps.

In another embodiment, the present invention relates to a cosmetic composition as defined above, said composition comprising, in a cosmetically acceptable medium:
aloe vera juice, the quantity of aloe vera juice being comprised from 42 to 74.2%, as a percentage by weight relative to the total weight of the composition,
pentylene glycol, the quantity of pentylene being from 3 to 5.8%, as a percentage by weight relative to the total weight of the composition,
at least one oil,
   said cosmetic composition being free of added water,
   the viscosity of said composition being from 45,000 and 80,000 cps.

In another embodiment, the present invention relates to a cosmetic composition as defined above, said composition comprising, in a cosmetically acceptable medium:
aloe vera juice, the quantity of aloe vera juice being comprised from 42 to 74.2%, as a percentage by weight relative to the total weight of the composition,
pentylene glycol,
at least one oil, the quantity of oil being comprised from 1 to 25% or from 2 to 25%,
as a percentage in weight relative to the total weight of the composition,
   said cosmetic composition being free of added water,
   the viscosity of said composition being from 45,000 and 80,000 cps.

In another embodiment, the present invention relates to a cosmetic composition as defined above, said composition comprising, in a cosmetically acceptable medium:
aloe vera juice,
pentylene glycol, the quantity of pentylene being comprised from 3 to 5.8%, as a percentage by weight relative to the total weight of the composition,
at least one oil, the quantity of oil being comprised from 1 to 25% or from 2 to 25%, as a percentage by weight relative to the total weight of the composition,
   said cosmetic composition being free of added water,
   the viscosity of said composition being from 45,000 to 80,000 cps.

In another embodiment, the present invention relates to a cosmetic composition as defined above, said composition comprising, in a cosmetically acceptable medium:
aloe vera juice, the quantity of aloe vera juice being comprised from 42 to 74.2%, as a percentage by weight relative to the total weight of the composition,
pentylene glycol, the quantity of pentylene being from 3 to 5.8%, as a percentage by weight relative to the total weight of the composition,
at least one oil, the quantity of oil being comprised from 1 to 25% or from 2 to 25%,
   as a percentage by weight relative to the total weight of the composition,
   said cosmetic composition being free of added water,
   the viscosity of said composition being comprised from 45,000 to 80,000 cps.

A fourth object of the present invention relates to the use of an association as defined above or of a cosmetic composition as defined above, for skin care, in particular of the face, hair, and/or or scalp, of a human, non-animal subject.

The association or cosmetic composition according to the present invention can be used as such as a cosmetic composition, or can serve as a basis for the development of a cosmetic composition.

Thus, it is possible to use the association or composition according to the invention to prepare cosmetic products such as: styling, deodorant, sunscreen, or facial care, by adding the respective basic elements of these products.

The cosmetic compositions according to the present invention can be prepared according to techniques commonly used in cosmetics.

The compositions are thus obtained by the prior preparation of at least two phases.

Each phase can include several ingredients, or just 1 ingredient.

Thus, typically and as an example:
a first phase comprising aloe vera juice stabilized with pentylene glycol, and possibly one or more ingredients and prepared to obtain phase A,
a second phase, comprising at least one oil, and optionally one or more ingredients and prepared, to obtain a phase B,
other phases (C, D, etc.) comprising ingredients to be included in the cosmetic composition are possibly prepared, then,
a phase comprising arginine is prepared,
   said phase B comprising said at least one oil is added to said phase A comprising aloe vera juice, then,
   the other possible phases are added, then
   the phase comprising arginine is added,
   the whole is homogenized to obtain the cosmetic composition.

In order to facilitate the homogenization and dissolution of the phases, it is, in certain cases, preferable to heat the phases beforehand, in particular to 70° C. Preheating is particularly desired when adding phase B to phase A, in order to melt the oily phase B.

Certain ingredients are preferably pre-solubilized in aloe vera juice stabilized with pentylene glycol, in order to facilitate the dissolution of the ingredient.

This is, for example, the case of flower acids, particularly red flower acids, which have low dissolution kinetics.

The following examples illustrate the invention, without limiting its scope.

### EXAMPLES

### Example A Stability Study

Several stabilizing agents in aloe vera juice have been analyzed:

### Aloe Vera Pure Juice Preserved in Leucidal (Leuconostoc/Radish Root Ferment Filtrate)

The juice was kept stable for 1 month at 4 temperatures: 4° C., 20° C., 40° C. and 50° C.
An intense coloring was observed on samples at 40° C. and 50° C.
Particles were observed on samples at 4° C./TA/40° C./50° C.
Deposition at the base was observed on samples at 40° C. and 50° C.
   Aloe Vera Pure Juice preserved with Potassium Sorbate/Sodium Benzoate

The juice is stable over time, under the conditions set out above, but the stabilizing agent is not compatible with the natural spirit of the invention.

### Aloe Vera Pure Juice Preserved in Pentylene Glycol

Formula remains colorless in stability tests.

Stability tests on the compositions of Examples 1 and 2 demonstrate that these compositions are also stable.

### Example B Sterilizing Power Tests

The objective of this test was to validate that aloe vera juice stabilized with pentylene glycol makes it possible to obtain compositions free of microorganisms.

### Preparation of the Sample and Inoculation of the Test Microorganisms

The samples were prepared under sterile conditions in microplates (2 mL well).

The samples were contaminated by three bacterial strains (Escherichia coli, Staphylococcus aureus and Pseudomonas aeruginosa) and two fungal strains (Candida albicans and Aspergillus niger), at a rate of approximately 3.30×10⁵ to 4.85×10⁵ cfu/mL for bacteria, 4.70×10⁴ cfu/mL for yeast and 3.62×10⁴ cfu/mL for mold.

At the end of the contamination, the samples were carefully mixed by suction-discharge cycles in order to ensure homogeneous distribution of the microorganism.

The whole was incubated at 22° C. for 14 days.

### Sampling and Counting of the Residual Microbial Population

Measurements of the residual contamination rate were carried out at 24 hours, 7 and 14 days for the five microbial strains. For bacteria, fungus and yeast, the contaminated sample was taken then deposited in serial dilutions in microtiter plates, in the presence of culture medium containing the neutralizer (GLYcoDiag formula adapted to simultaneous neutralization at the stage of counting) and a reagent revealing dehydrogenase activity.

### Results

The microbial population measurements taken at each sampling time are expressed in colony forming units/mL.

TABLE 1
Results of sterilizing power tests

| | | number of colony- forming units after | | |
|---|---|---|---|---|
| Strains | Inoculum* | 24 h | 7 days | 14 days |
| Candida albicans ATCC10231 | 4.70E+04 | 0.00E+00 | 0.00E+00 | 0.00E+00 |
| Escherichia coli ATCC8739 | 3.30E+05 | 0.00E+00 | 0.00E+00 | 0.00E+00 |
| Staphylococcus aureus ATCC6538 | 3.50E+05 | 0.00E+00 | 0.00E+00 | 0.00E+00 |
| Pseudomonas aeruginosa | 4.85E+05 | 0.00E+00 | 0.00E+00 | 0.00E+00 |
| ATCC9027 | | | | |
| Aspergillus brasiliensis | 3.62E+04 | 0.00E+00 | 0.00E+00 | 0.00E+00 |
| ATCC16404 | | | | |

*cfu/ml

The compositions according to Examples 1 and 2 are also sterile.

### Example 1 Hair Care Formula

The composition in Table 2 was manufactured according to the following procedure.

Solagum™ Tara was dissolved in glycerin, then added to aloe vera.

The whole was heated to 75° C. then G AminoSensyl™ HC was added to obtain phase A. A mixture of SustOleo™ BA and argan oil was heated to 75° C. to obtain the molten phase B.

The two phases A and B were mixed then stirred at 1,200 rpm for 10 minutes.

The mixture thus obtained was cooled to 30° C., then phases C, D and E were added sequentially to obtain the composition in Table 2.

TABLE 2
Composition of a hair care formula

| Phase | name | Commercial name | INCI name | % |
|---|---|---|---|---|
| A | Aloe vera and | Aloe vera pur organic juice | Aloe Barbadensis Leaf | 71.899 |
| | pentylène | 1:1 fair trade PentG (7%) | Juice, Pentylene Glycol | |
| | glycol | | | |
| | Tara gum | Solagum ™ Tara | Caesalpinia Spinosa Gum | 1 |
| | Glycerin | Glycerin 4803 | Glycerin | 2 |
| | Brassicyl | AminoSensyl ™ HC | Brassicyl Valinate Esylate | 12 |
| | Valinate | | (and) Brassica Alcohol | |
| | Esylate and | | (and) calcium gluconate | |
| | calcium | | | |
| | gluconate and | | | |
| | Brassica | | | |
| | Alcohol | | | |
| B | Brassica | SustOleo ™ BA | Brassica Alcohol | 5 |
| | Alcohol | | | |
| | Argan Oil | Argan Oil | argania Spinosa Kernel Oil | 5 |
| C | Aloe vera et | Aloe vera pur jus bio 1:1 | Aloe Barbadensis Leaf | 1 |
| | pentylène | equitable PentG (7%) | Juice, Pentylene Glycol | |
| | glycol | | | |
| | red flower | red flower acids ES (001233) | Hibiscus sabdariffa flower | 0.1 |
| | acids | | extract, maltodextrin | |
| D | Coconut milk | Coconut milk fragrance 2 | Fragrance | 2 |
| | fragrance | NATFLOR RT4683 | | |
| E | L- arginine | L- ARGININE | Arginine | 0.001 |

| | |
|---|---|
| Total | 100 |

### Example 2 Hair Care Formula

The hair care formula in Table 3 was prepared in the same way as the formula in Example 1.

TABLE 3
Composition of a hair care formula

| Name | INCI | % |
|---|---|---|
| Aloe vera and pentylene | Aloe Barbadensis Leaf Juice, | 72.899 |
| glycol | Pentylene Glycol | |
| Sodium Phytate | Sodium Phytate | 0.1 |
| Brassicyl valinate estylate and | Brassicyl Valinate Esylate | 12 |
| Calcium gluconate and | (and) Brassica Alcohol (and) | |
| Brassica alcohol | caclium gluconate | |
| Brassica alcohol | Brassica Alcohol | 5 |
| Argan oil | Argania Spinosa Kernel Oil | 5 |
| Tara gum | Caesalpinia Spinosa Gum | 1 |
| Glycerine | Glycerin | 2 |
| | Fragrance | 2 |
| Arginine | Arginine | 0.001 |
| | Total | 100 |

### Example 3 Hair Care Formula

The hair care formula in Table 4 was prepared in the same way as the formula in Example 1.

TABLE 4
Composition of a hair care formula

| name | INCI | % |
|---|---|---|
| Aloe vera and pentylene | Aloe Barbadensis Leaf Juice, | 74.999 |
| glycol | Pentylene Glycol | |
| Brassicyl valinate estylate and | Brassicyl Valinate Esylate | 12 |
| Calcium gluconate and | (and) Brassica Alcohol (and) | |
| Brassica alcohol | caclium gluconate | |
| Glyceryl stearate | Glyceryl stearate | 3 |
| Argan oil | Argania Spinosa Kernel Oil | 5 |
| Tara Eraser | Caesalpinia Spinosa Gum | 1 |
| Glycerine | Glycerin | 2 |
| Fragrance | Fragrance | 2 |
| Arginine | Arginine | 0.001 |
| | Total | 100 |

### Example 4 Hair Care Formula

The hair care formula in Table 5 was prepared in the same way as the formula in Example 1.

TABLE 5
Composition of a hair care formula

| Phase | name | trading name | INCI name | % |
|---|---|---|---|---|
| A | Aloe vera | Pure organic aloe vera juice | Aloe Barbadensis Leaf | 78.92425 |
| | and | 1:1 equitable PentG (7%) | Juice, Pentylene Glycol | |
| | pentylene | | | |
| | glycol | | | |
| | Tara gum | Solagum ™ Tara | Caesalpinia Spinosa Gum | 0.75 |
| | Glycerine | Glycerine 4803 | Glycerin | 1.5 |
| | Brassicyl | AminoSensyl ™ HC | Brassicyl Valinate Esylate | 9 |
| | valinate | | (and) Brassica Alcohol | |
| | estylate and | | (and) caclium gluconate | |
| | Calcium | | | |
| | gluconate | | | |
| | and Brassica | | | |
| | alcohol | | | |
| B | Brassica | SustOleo ™ BA | Brassica Alcohol | 3.75 |
| | alcohol | | | |
| | Argan oil | Argan oil | argania Spinosa Kernel Oil | 3.75 |
| C | Aloe vera | Pure organic aloe vera juice | Aloe Barbadensis Leaf | 0.75 |
| | and | 1:1 equitable PentG (7%) | Juice, Pentylene Glycol | |
| | pentylene | | | |
| | glycol | | | |
| | Red flower | Red flower acids ES (001233) | Hibiscus sabdariffa flower | 0.075 |
| | acids | | extract, maltodextrin | |
| D | Coconut | COCONUT MILK | Fragrance | 1.5 |
| | milk | FRAGRANCE 2 NATFLOR | | |
| | fragrance | RT4683 | | |
| E | L- arginine | L- ARGININE | Arginine | 0.00075 |

| | |
|---|---|
| Total | 100 |

### Example 5 Facial Care Formula With Prebiotic

The composition in Table 6 was manufactured.

TABLE 6
Composition of a facial care formula

| name | trading name | INCI name | % |
|---|---|---|---|
| Aloe vera and | Aloe Vera pure organic juice | ALOE BARBADENSIS | 73.0499 |
| pentylene | 1:1 equitable PentG (7%) | LEAF JUICE, | |
| glycol | | PENTYLENE GLYCOL | |
| Xanthan gum | XGF FFCS- PC (XANTHAN | XANTHAN GUM | 1 |
| | GUM) | | |
| Glyceryl | SustOleo ™ GMS | GLYCERYL STEARATE | 3 |
| stearate | | | |
| hydrogenated | EMULMETIK ™ 320 | HYDROGENATED | 1 |
| lecithin | | LECITHIN | |
| Argan oil | ARGAN OIL | ARGANIA SPINOSA | 3 |
| | | KERNEL OIL | |
| Sunflower oil | DESODO ORGANIC | HELIANTHUS ANNUS | 16 |
| | SUNFLOWER OIL | SEED OIL | |
| Vitamin E | Covi- ox ® T90 EU C | TOCOPHEROL | 0.5 |
| Citric acid | Anhydrous citric acid | CITRIC ACID | 0.0001 |
| Arginine | L- ARGININE C- GRADE | ARGININE | 1.45 |
| α- glucan | Bioecolia ® | ALPHA GLUCAN | 1 |
| oligosaccharide | | OLIGOSACCHARIDE | |

| | |
|---|---|
| Total | 100 |

### Example 6 Prebiotic-Free Facial Treatment Formula

The composition in Table 7 was manufactured according to the following procedure:

Phase A was heated to 70-75° C. Phase B was dispersed in phase A for 15 min, with vigorous stirring, using a deflocculator. Phase C was heated to 70-75° C., then added to the mixture of phases A and B. The mixture thus obtained was emulsified for 20 min using a deflocculator.

Then, phase D was added at 40° C., followed by phase E, to obtain the composition in Table 7.

TABLE 7
Composition of a facial care formula

| Phase | name | trading name | INCI name | % |
|---|---|---|---|---|
| A | Aloe vera and | Aloe Vera pure organic juice | ALOE BARBADENSIS | 71.85 |
| | pentylene | 1:1 equitable PentG (7%) | LEAF JUICE, | |
| | glycol | | PENTYLENE GLYCOL | |
| B | Xanthan gum | XGF FFCS- PC (XANTHAN | XANTHAN GUM | 1 |
| | | GUM) | | |
| C | Glyceryl | SustOleo ™ GMS | GLYCERYL STEARATE | 3 |
| | stearate | | | |
| | hydrogenated | EMULMETIK ™ 320 | HYDROGENATED | 1 |
| | lecithin | | LECITHIN | |
| | Argan oil | ARGAN OIL | ARGANIA SPINOSA | 5 |
| | | | KERNEL OIL | |
| | Sunflower oil | DESODO ORGANIC | HELIANTHUS ANNUS | 16 |
| | | SUNFLOWER OIL | SEED OIL | |
| D | Vitamin E | Covi- ox ® T90 EU C | TOCOPHEROL | 0.5 |
| E | Citric acid | Anhydrous citric acid | CITRIC ACID | 0.01 |
| | Arginine | L- ARGININE C- GRADE | ARGININE | 1.64 |

| | |
|---|---|
| Total | 100 |

Table 7 Composition of a facial care formula

A composition was also prepared by replacing argan oil with plum oil.

### Example 7 Facial Care Formula With Prebiotic

The composition in Table 8 was manufactured according to the following procedure:

Phase A was heated to 70-75° C. Phase B was dispersed in phase A for 15 min, with vigorous stirring, using a deflocculator. Phase C was heated to 70-75° C., then added to the mixture of phases A and B. The mixture thus obtained was emulsified for 20 min using a deflocculator.

Then, phases D and E were added at 30° C., followed by phase F, to obtain the composition of Table 8

TABLE 8
Composition of a facial care formula

| Phase | name | trading name | INCI name | % |
|---|---|---|---|---|
| A | Aloe vera and | Aloe Vera pure organic juice | ALOE BARBADENSIS | 73.87 |
| | pentylene | 1:1 equitable PentG (7%) | LEAF JUICE, | |
| | glycol | | PENTYLENE GLYCOL | |
| B | Xanthan gum | XGF FFCS- PC (XANTHAN | XANTHAN GUM | 1 |
| | | GUM) | | |
| C | Glyceryl | SustOleo ™ GMS | GLYCERYL STEARATE | 3 |
| | stearate | | | |
| | hydrogenated | EMULMETIK ™ 320 | HYDROGENATED | 1 |
| | lecithin | | LECITHIN | |
| | Argan oil | ARGAN OIL | ARGANIA SPINOSA | 3 |
| | | | KERNEL OIL | |
| | Sunflower oil | DESODO ORGANIC | HELIANTHUS ANNUS | 16 |
| | | SUNFLOWER OIL | SEED OIL | |
| D | Vitamin E | Covi- ox ® T90 EU C | TOCOPHEROL | 0.5 |
| F | Citric acid | Anhydrous citric acid | CITRIC ACID | 0.03 |
| | Arginine | L- ARGININE C- GRADE | ARGININE | 0.6 |
| E | α- glucan | Bioecolia ® | ALPHA GLUCAN | 1 |
| | oligosaccharide | | OLIGOSACCHARIDE | |

| | |
|---|---|
| Total | 100 |

### Example 8 Charcoal Hair Care Formula

The hair care formula in Table 9 was prepared in the same way as the formula in Example 1.

TABLE 9
Composition of a formula of the Charcoal hair care formula

| Phase | name | trading name | INCI name | % |
|---|---|---|---|---|
| A | Aloe vera and | Pure organic aloe vera juice | Aloe Barbadensis Leaf | 74.987 |
| | pentylene | 1:1 equitable PentG (7%) | Juice, Pentylene Glycol | |
| | glycol | | | |
| | Tara gum | Solagum ™ Tara | Caesalpinia Spinosa Gum | 0.753 |
| | Glycerine | Glycerine 4803 | Glycerin | 1.504 |
| | Brassicyl | AminoSensyl ™ HC | Brassicyl Valinate Esylate | 9.026 |
| | valinate | | (and) Brassica Alcohol | |
| | estylate and | | | |
| | Brassica | | | |
| | alcohol | | | |
| B | Brassica | SustOleo ™ BA | Brassica Alcohol | 3.769 |
| | alcohol | | | |
| | Argan oil | Argan oil | argania Spinosa Kernel Oil | 3.761 |
| C | Aloe vera and | Pure organic aloe vera juice | Aloe Barbadensis Leaf | 2 |
| | pentylene | 1:1 equitable PentG (7%) | Juice, Pentylene Glycol | |
| | glycol | | | |
| | Vegetable | Powdered charcoal | Charcoal powder | 0.2 |
| | charcoal | | | |
| | powder | | | |
| D | α- glucan | Bioecolia ® | ALPHA GLUCAN | 1 |
| | oligosaccharide | | OLIGOSACCHARIDE | |
| | Propanediol | Crinipan PMC green | Propanediol caprylate | 1 |
| | caprylate | | | |
| | Herbs & tea | Herbs & tea | fragrance | 2 |

| | |
|---|---|
| Total | 100 |

### Example 9 Face Cream Formula

TABLE 10
Composition of a face cream formula

| Phase | Nom commercial | Nom INCI | % |
|---|---|---|---|
| A | Pure organic aloe vera juice 1:1 | Aloe Barbadensis Leaf | 70-80 |
| | equitable PentG (7%) | Juice, Pentylene Glycol | |
| B | XGF FFCS- PC (XANTHAN GUM) | Xanthan gum | 0.5-1 |
| | Glycerine 4803 | Glycerin | 1-5 |
| C | Sustoleo GMS SE | Glyceryl Stearate SE | 3-10 |
| | Argan oil | Argania Spinosa Kernel | 3-10 |
| | | Oil | |
| | LexFeel Natural | Heptyl Undecylenate | 3-8 |
| D | Coviox T90 EU C | Tocopherol | 0.2-1 |
| | BIOECOLIA | Alpha glucan | 0.5-2 |
| | | oligosaccharide | |
| | Parfum | Parfum | 0.3-1 |
| | Peptiyouth | s- Pea Oligopeptide- 1 SP | 0.003-0.01 |
| E | L- ARGININE | Arginine | 0.2-01 |

The face cream formula in table 10 was prepared as follows:
1/Heat phase A and B to 70-75° C.
2/Mix the xanthan gum in the glycerin, then add in phase A.
3/When phase C has melted, emulsify the fatty phase in the aqueous phase.
4/Stir for 10 minutes then turn off the heat.
5/Stir until cool. Plunge in a cold water bath to speed up cooling.
6/At 30° C., add the ingredients of phase D.
7/Adjust the pH with phase E.

### Example 10 Ceramide Balm Formula

TABLE 11
Composition of a Ceramide Balm Formula

| Phase | Trade name | INCI name | % |
|---|---|---|---|
| A | Pure organic aloe vera juice | Aloe Barbadensis Leaf Juice, Pentylene | 60-80 |
| | 1:1 equitable PentG (7%) | Glycol | |
| B | Glycerine 4803 | Glycerin | 1-5 |
| | Solagum Tara | Caesalpinia Spinosa Gum | 1-2 |
| C | AminoSensyl HC | Brassicyl Valinate Esylate (and) Brassica | 5-12 |
| | | Alcohol | |
| D | SustOleo BA | Brassica Alcohol | 2-5 |
| | CosmeGreen ES 1822+ | Arachidyl/Behenyl Betainate Esylate (and) | 5-12 |
| | | Arachidyl/Behenyl Alcohol | |
| | Argan oil | Argania Spinosa Kernel Oil | 2-10 |
| | LexFeel Natural | Heptyl Undecylenate | 3-8 |
| E | Fragrance | Fragrance | 0.3-2 |
| | C + eRAMIDE | Triticum vulgare (wheat) seed extract | 0.3-1 |
| F | L- ARGININE | Arginine | 0.01-1 |

The ceramide balm formula in table 11 was prepared as follows:
1/Heat phases A and B to 75° C.
2/Mix Solagum Tara in glycerin, then add to phase A.
3/When phase A is >70° C., add Aminosensyl HC.
4/When phase D has melted, emulsify the fatty phase in the aqueous phase.
5/Stir for 10 minutes then turn off the heat.
6/Stir until cool. Plunge in a cold water bath to speed up cooling.
7/At 30° C., add the ingredients of phase E.
8/Adjust the pH with phase F.

### Example 11 Hair Care Formula

The hair care formula in Table 12 was prepared in the same wav as the formula in Example 1.

TABLE 12
Composition of a hair care formula

| Phase | name | trading name | INCI name | % |
|---|---|---|---|---|
| A | Aloe vera | Aloe vera pure organic juice | Aloe Barbadensis Leaf | 74.03265 |
| | | 1:1 fair trade | Juice, Pentylene Glycol | |
| | pentylene | PentG | Pentylene glycol | 5.57235 |
| | glycol | | | |
| | Tara gum | Solagum ™ Tara | Caesalpinia Spinosa Gum | 0.753 |
| | Glycerine | Glycerine 4803 | Glycerin | 1.505 |
| | Brassicyl | AminoSensyl ™ HC | Brassicyl Valinate Esylate | 9.026 |
| | valinate | | (and) Brassica Alcohol | |
| | estylate and | | (and) caclium gluconate | |
| | Calcium | | | |
| | gluconate | | | |
| | and Brassica | | | |
| | alcohol | | | |
| B | Brassica | SustOleo ™ BA | Brassica Alcohol | 3.769 |
| | alcohol | | | |
| | Argan oil | Argan oil | argania Spinosa Kernel Oil | 3.761 |
| C | Red flower | Red flower acids ES (001233) | Hibiscus sabdariffa flower | 0.076 |
| | acids | | extract, maltodextrin | |
| D | Coconut | COCONUT MILK | Fragrance | 1.504 |
| | milk | FRAGRANCE 2 NATFLOR | | |
| | fragrance | RT4683 | | |
| E | L- arginine | L- ARGININE | Arginine | 0.001 |

| | |
|---|---|
| Total | 100 |

A usability test under dermatological control of the hair mask was carried out over a period of 21 days under normal conditions of use.

The hair mask was tested on 24 women aged 21 to 69 with dry and damaged hair for 21 days.

According to the dermatologist's assessment, the hair mask presented excellent dermatological tolerance on dry hair and scalp, as well as excellent cosmetic acceptability, with 100% favorable opinions.

An evaluation of clinical and dermatological tolerance was carried out.

No clinical signs of intolerance were observed.

No sensation of discomfort was reported.

Items relating to product effectiveness (positive responses ³ 74% *):
The hair is strengthened
The product does not weigh down the hair
Hair is shiny
The product has an anti-frizz effect on the hair
Hair is easy to detangle
Hair is nourished
Hair is soft
Hair is protected
Hair is soft
Hair is disciplined
The product is adapted to your hair type
Hair is hydrated

1.-19. (canceled)

20. An association consisting of the following 2 components:
aloe vera juice stabilized with pentylene glycol, and
at least one oil,
said association being free of added water.

21. The association according to claim 20, consisting of the following 3 components:
aloe vera juice stabilized with pentylene glycol,
arginine, and
at least one oil,
said association being free of added water.

22. The association according to claim 20, in which the at least one oil is chosen from:
jojoba oil,
sweet almond oil,
oil of bitter almond,
argan oil,
sesame oil,
camellia oil,
safflower oil,
moringa oil,
castor oil,
coconut oil,
apricot oil,
flaxseed oil,
pomegranate oil,
grapeseed oil,
prickly pear oils,
marula oil,
evening primrose oil,
avocado oil,
raspberry oil,
borage oil,
olive oil,
neem oil,
sunflower oil,
walnut or hazelnut oil,
amla oil,
plum oil,
coffee oil,
hemp oil,
rice bran oil,
vanilla macerate,
oil powder, and
fermented oil,
and a mixture thereof.

23. The association according to claim 20, in which the quantity of aloe vera juice stabilized with pentylene glycol is from 60 and 95%, as a percentage by weight relative to the total weight of the association, and/or
in which the quantity of Arginine is from 0.00015 and 5%, as a percentage by weight relative to the total weight of the association, and/or
in which the total quantity of oil is from 3 and 40%, as a percentage by weight relative to the total weight of the association.

24. A cosmetic composition, comprising the association according to claim 20, in a cosmetically acceptable medium,
said cosmetic composition being free of added water.

25. The cosmetic composition according to claim 24, comprising, in a cosmetically acceptable medium, the following 2 components:
aloe vera juice stabilized with pentylene glycol, and
at least one oil,
said cosmetic composition being free of added water,
or
comprising, in a cosmetically acceptable medium, the following 3 components:
aloe vera juice stabilized with pentylene glycol,
arginine, and
at least one oil,
said cosmetic composition being free of added water,
or
comprising, in a cosmetically acceptable medium, the following 4 components:
aloe vera juice stabilized with pentylene glycol,
arginine,
at least one oil, and
a prebiotic, in particular glucan oligosaccharide,
said cosmetic composition being free of added water,
said cosmetic composition being a composition for the care of the skin,
or
comprising, in a cosmetically acceptable medium, the 4 following components:
aloe vera juice stabilized with pentylene glycol,
arginine,
at least one oil, and
a flower acid,
said cosmetic composition being free of added water,
said cosmetic composition being a composition for hair care.

26. The cosmetic composition according to claim 24, in which the quantity of aloe vera juice stabilized with pentylene glycol is from 45 to 80%, as a percentage by weight relative to the total weight of the composition, and/or
in which the quantity of Arginine is from 0.0001 to 2%, as a percentage by weight relative to the total weight of the composition, and/or
in which the total quantity of oil is from 2 to 25%, as a percentage by weight relative to the total weight of the composition.

27. The cosmetic composition according to claim 24, in which the quantity of aloe vera juice is from 42 to 74.2%.

28. The cosmetic composition according to claim 24, in which the quantity of pentylene glycol is from 3 to 5.8%.

29. The cosmetic composition according to claim 24, said composition comprising, in a cosmetically acceptable medium:
aloe vera juice, the quantity of aloe vera juice is from 42 to 74.2%, as a percentage by weight relative to the weight total of the composition,
pentylene glycol,
at least one oil, the quantity of oil is from 1 to 25% or from 2 to 25%, as a percentage by weight relative to the total weight of the composition,
said cosmetic composition being free of added water.

30. The cosmetic composition according to claim 24, in which the composition further comprises at least one active compound chosen from:
a surfactant,
glycerin,
cannabidiol,
flower acid,
Zinc PCA,
a pigment,
vegetable charcoal,
an antioxidant, or vitamin C, or one of its derivatives,
niacinamide,
powdered tea or matcha green tea, fermented tea, powdered kombucha tea, centella asiatica, or madecassoside,
sugars, or xylitol, rhamnose, or fructooligosaccharides, apple extract powder
a protein, or a pea protein, soy protein, wheat protein, or rice protein,
sweet almond milk, or one or more of its components, or prunus dulcis, rice syrup or acacia fiber,
a fragrance, or an apricot almond fragrance, a coconut milk fragrance,
ghassoul,
white clay, in particular kaolin,
hyaluronic acid or one of these derivatives, or sodium hyaluronate, lactic acid, vanilla macerate,
maltodextrin,
bamboo powder,
spirulina,
phycocianin,
fermented mushrooms,
guarana powder,
caffeine,
acerola,
chamomile,
ayurvedic powders,
vitamins, or vitamin B3, or vitamin B5,
glycosphingolipids and glycolipids,
a prebiotic, exopolysaccharides, postbiotics, or lactobacilus ferment,
pyroctolamine,
piroctone olamine,
an extract of camellia seeds,
powder of cornflower,
chamomile flower extract,
calcium gluconate,
collagen,
elastin,
Manuka honey,
vinegar, or rice vinegar,
sodium phytate,
ceramide,
probiotic,
marshmallow powder, and
peptides, or quinoa peptides, and
a mixture of these ingredients.

31. The cosmetic composition according to claim 24, in which said composition comprises:
aloe vera juice,
pentylene glycol,
tara gum,
glycerin,
brassicyl valinate esylate,
Brassica alcohol,
calcium gluconate,
argan oil,
red flower acids,
the scent of coconut milk, and
arginine,
or in which said composition comprises:
aloe vera juice,
pentylene glycol,
xanthan gum,
glyceryl stearate,
hydrogenated lecithin,
argan oil,
sunflower oil,
vitamin E,
citric acid,
a-glucan oligosaccharide, and
arginine,
or in which said composition comprises:
aloe vera juice,
pentylene glycol,
Sodium phytate
brassicyl valinate esylate,
Brassica alcohol,
calcium gluconate,
argan oil,
tara gum,
glycerin,
arginine,
or in which said composition comprises:
aloe vera juice,
pentylene glycol,
glyceryl stearate,
brassicyl valinate esylate,
Brassica alcohol,
calcium gluconate,
argan oil,
tara gum,
glycerin,
arginine.

32. The cosmetic composition according to claim 24, said composition being in the form of a cream, or a melting cream.

33. The cosmetic composition according to claim 24, said composition being free of synthetic preservatives.

34. The cosmetic composition according to claim 24,said composition being free of synthetic and natural preservatives, other than pentylene glycol.

35. The cosmetic composition according to claim 24, said composition exhibiting microbial stability.

36. The cosmetic composition according to claim 24, said composition exhibiting an absence of rancidity of the oil.

37. A method for cosmetically treating the skin of a subject comprising applying on the face, hair, and/or scalp of the subject, the association according to claim 1 or a cosmetic composition comprising the association, in a cosmetically acceptable medium, said cosmetic composition being free of added water.
